(19) United States
(12) Patent Application Publication
(10) Pub. No.: US 2021/0131229 A1
(43) Pub. Date: May 6, 2021

(54) WAX CUTTING TOOL FOR REMOVING WAX FROM AN OIL WELL TUBING

(71) Applicant: EXPRO NORTH SEA LIMITED, Aberdeen (GB)

(72) Inventor: Andre Alexander MINNIS, Yarmouth (GB)

(73) Assignee: Expro North Sea Limited, Aberdeen (GB)

(21) Appl. No.: 17/041,893

(22) PCT Filed: Mar. 8, 2019
(86) PCT No.: PCT/EP2019/055932

§ 371 Date: Sep. 25, 2020

(30) Foreign Application Priority Data
Mar. 27, 2018 (GB) 1804927.0

(51) Int. Cl. E21B 37/02; A61B 17/3207

(52) U.S. Cl. CPC E21B 37/02; A61B 17/320725

(57) ABSTRACT

A wax cutter tool for removing wax from an internal wall of an oil well tubing is provided that includes a central wax cutter tool body and one or more wax cutter elements. The central wax cutter tool body is configured to be attached to an elongate member for deployment down an oil well tubing. The wax cutter element extends radially outwards from the central wax cutter tool body and has an outer working surface for engaging the internal wall of the oil well tubing. The one or more wax cutter elements are flexible and may be compressed when deployed within the oil well tubing such that the outer diameter of the outer working surface of the one or more wax cutter elements is reduced to match an inner diameter of the oil well tubing.

This application claims priority to PCT Patent Appln. No. PCT/EP2019/055932 filed Mar. 8, 2019, which claims priority GB Patent Appln. No. 1804927.0 filed Mar. 27, 2018, which are herein incorporated by reference.

### BACKGROUND OF THE INVENTION

### 1. Technical Field

The present specification relates to a wax cutting tool, system, and method for removing wax from an internal wall of an oil well tubing.

### 2. Background Information

One problem associated with oil well tubing components, such as production tubulars and completion equipment, is the build-up of paraffin and soft wax deposits on the internal wall of the oil well tubing. Such wax deposits can impede sub-surface equipment from passing freely through the tubing and will cause a smaller flow area, which in turn reduces production yield of oil.

To solve these problems, tools known variously as wax cutters, paraffin scrapers, or gauge cutters are provided to remove paraffin and soft wax deposits from the internal wall of the oil well tubing. Such tools are attached to an elongate member, such as a wireline tool string assembly, for deployment down an oil well tubing. In this regard, it will be noted that “wireline” is a term of art for well-intervention operations conducted using single-strand or multi-strand wire or cable for interventions in oil and gas wells. Furthermore, the term “oil well tubing” is used herein as a generic term to mean oil and/or gas well tubing.

The wax cutting tools are commonly fixed assemblies with fixed diameters selected to match the smallest inner drift diameter of the oil well tubing to be cleaned of wax. However, one problem with such tools is that they can become hung up on edges, protrusions, or impediments within the oil well tubing requiring the use of a jar which delivers an impact load to free the wax cutting tool. Such wax cutting tools generate no jarring or impact force themselves and thus require the use of a separate jar. In this regard, it will be noted that “jar” is a term of art for a device used downhole to deliver an impact load to another downhole component, especially when that component is stuck or hung up. An alternative to the provision of fixed diameter devices would be to provide a wax cutting tool having a diameter which can be varied to fit an inside diameter of an oil well tubing for a particular application. However, even then, such a configuration would have similar problems to those outlined above for the fixed diameter tools. That is, such tools are prone to catching on edges, protrusions, or impediments within the oil well tubing requiring the use of a separate jar which delivers an impact load to free the wax cutting tool. Again, such wax cutting tools produce no jarring or impact force themselves within the wax cutting tool assembly to free themselves when caught on edges, protrusions, or impediments within the oil well tubing.

Of course, one way to alleviate the problem of the device getting stuck within the tubing is to select or fix the diameter of the tool to be less than the inner diameter of the tubing such that there is sufficient clearance to avoid small edges protrusions, or impediments. However, this approach results in a significant quantity of wax remaining on the interior wall of the tubing.

It is an aim of the present invention to address the aforementioned problems.

### SUMMARY OF THE INVENTION

According to one configuration as described herein, there is provided a wax cutter tool for removing wax from an oil well tubing, the wax cutter tool comprising: a central wax cutter tool body, the central wax cutter tool body being configured to be attached to an elongate member for deployment down an oil well tubing; and one or more wax cutter elements extending radially outwards from the central wax cutter tool body, the one or more wax cutter elements having an outer working surface for engaging the internal wall of the oil well tubing in use, the outer working surface of the one or more wax cutter elements defining an outer diameter of the wax cutter tool, wherein the one or more wax cutter elements are flexible thereby allowing compression of the one or more cutter elements in use when deployed within the oil well tubing such that the outer diameter of the outer working surface of the one or more wax cutter elements is reduced to match an inner diameter of the oil well tubing when inserted into the oil well tubing to remove wax from the inner wall of the oil well tubing.

Providing flexible cutter elements which can be compressed enables the cutter elements to provide a good fit to the interior wall of the oil well tubing when deployed within the tubing improving removal of wax deposits on the interior of the tubing and oil well subassemblies. In operation the cutter elements can provide a small radial force on the internal wall of the oil well tubing which is sufficient to maintain a good contact between the cutter and the wall without causing the cutter to be prevented from moving along the tubing. Furthermore, the cutter elements can flex to account for edges, protrusions, or impediments and thus alleviate problems of the tool becoming stuck. Further still, as the cutter elements flex under loading the stored energy can then release providing an integrated jar or impact force to free the tool should it become stuck. That is, the wax cutting tool allows for full bore diameter excavation of the wax back to the tubing wall and also provides an integrated jar or impact force mechanism should the tool become help up by any well resistance or well debris.

In one configuration as described herein, the working surface of the one or more wax cutter elements is flexible to enable compression of the one or more cutter elements in use when deployed within the oil well tubing. However, it is also envisaged that in an alternative configuration the working surface of the one or more wax cutter elements can be rigid and the rigid working surface can be mounted to the central wax cutter tool body by one or more flexible mounting components to enable compression of the one or more cutter elements in use when deployed within the oil well tubing. For example, rigid blade components could be mounted to the central wax cutter tool body via a flexible (e.g. spring) coupling to provide a flexible/compressible functionality. However, such multi-component cutter elements are likely to be more complex and also reduce tool fluid bypass and thus are less desirable for certain applications. A simpler approach is to form each cutter element utilizing a single component which can be flexed and compressed in use.

The one or more cutter elements may include a wire blade or a plurality of wire blades disposed along the central wax cutter tool body. In one configuration as described herein, the one or more cutter elements form a helical or spiral wire blade structure. This can be formed by a single wire or a plurality of wires each forming a segment of the helical structure. Such a configuration is useful for cutting wax from the internal wall of a tubing and it has the ability to coil and change diameter while maintain pressure on the inside wall of the tubing during cutting. Furthermore, such a tool configuration incorporates cutting action, tube sizing, and impact force functionality all within the same assembly.

According to another feature of the wax cutter tool as described herein, one or more fluid paths are provided between the central wax cutter tool body and the outer working surface of the one or more wax cutter elements to enable fluid to flow though the wax cutter tool when it is deployed in an oil well tubing. The wax cutter tool has a total cross-sectional area defined by the outer working surface, and the one or more fluid paths may have a cross-sectional area which forms at least 30%, 50%, or 75% of the total cross-sectional area of the wax cutter tool. That is, the tool has a low physical cross-sectional area to reduce flow lift from the well and increase fluid bypass in the well. This also assists the clearing of wax from the cutter. The previously described helical wire cutter structure is particularly useful in providing such a low physical cross-sectional area to aid fluid bypass and removal of wax.

According to another feature of the wax cutter tool as described herein, the central wax cutter tool body is configured to enable free rotation of the one or more wax cutter elements when the wax cutter tool is coupled to an elongate member and deployed down an oil well tubing. Enabling a free rotation of the one or more wax cutter elements relative to the elongate (e.g. wireline) member and the oil well tubing can alleviate problems of the tool becoming stuck on edges, protrusions, or impediments. Furthermore, the rotating action can aid cutting and removal of wax from the interior wall of the oil well tubing. Further still, the rotational action is provided without having to actively rotating the entire elongate member extending through the oil well tubing. In one such configuration, the central wax cutter body comprises a tubular member and a central rod extending through the tubular member, wherein the tubular member is rotatable around the central rod, and wherein the one or more wax cutter elements are mounted on the tubular member.

The central wax cutter tool body can also be configured to enable free translational movement of the one or more wax cutter elements when the wax cutter tool is coupled to an elongate member and deployed down an oil well tubing. That is, the one or more wax cutter elements are free to move upwards or downwards relative to the elongate member and the oil well tubing, at least within a defined length bounded by an upper and lower stopper. Again, this can be useful to alleviate problems of the tool becoming stuck on edges, protrusions, or impediments, especially when provided in combination with the previously described rotational mounting. In one such configuration, the central wax cutter body comprises a tubular member and a central rod extending through the tubular member as previously described, wherein the tubular member is movable up and down the central rod, and the central rod includes an upper stopper and a lower stopper to limit the extent of translational movement of the tubular member. The upper and/or lower stoppers may be attachable and detachable from the central rod to aid in construction, disassembly, and cleaning and replacement of the tool components.

According to another feature of the wax cutter tool as described herein, the central wax cutter tool body comprises a plurality of protrusions providing attachment points for the cutter elements. This configuration is particular useful when the central wax cutter tool body is configured as previously described to have a tubular member and a central rod extending through the tubular member such that the tubular member can rotate around the central rod and translate along the central rod. To provide smooth rotational/translational motion of the tubular member it must be ensured that the cutter element attachments do not adversely compromise a smooth interface between the central rod and the tubular member. As such, protrusions or wings on an outer surface of the tubular member can be provided to function as attachment points for the cutter elements. The plurality of protrusions can be shaped with a radius of curvature or a beveled angle to alleviate the possibility of the protrusions catching on any edges, protrusions, or impediments, within the oil well tubing.

In one configuration the tubular member has a substantially circular cross-section, optionally with protrusions to provide mounting/attachment points for the one or more cutter elements. Alternatively, the tubular member may have a polygonal cross-section such as a square cross-section. In this arrangement, corners of the polygonal tubular member can provide attachment points for the one or more cutter elements. For example, holes can be provided through the corners of a polygonal tubular member for attaching the one or more cutter elements. A polygonal tubular member can have a reduced contact surface area with a central cylindrical rod on which it is mounted and thus result in a reduction in friction between the central rod and the tubular member. A polygonal tubular member can also increase the bypass space for wax and/or fluid to flow through/around/passed the wax cutter tool when disposed within an oil well tubing.

Another aspect of the invention provides an oil well wax cutter system comprising: an elongate member for deployment down an oil well tubing; and a wax cutter tool as described herein which is configured to be attached to the elongate member for deployment down an oil well tubing.

The system may also include the oil well tubing. In such a system, the outer diameter of the wax cutter tool can be equal to or larger than the inner diameter of the oil well tubing in a fully expanded form and is compressible to be inserted into the oil well tubing.

Yet another aspect of the invention provides a method of removing wax from an internal wall of an oil well tubing, the method comprising: connecting the wax cutter tool as described herein to an elongate member; inserting the wax cutter tool into an oil well tubing; and moving the wax cutter tool along the oil well tubing to remove wax from an inner wall of the oil well tubing.

### BRIEF DESCRIPTION OF THE DRAWINGS

Embodiments of the present invention are described by way of example only with reference to the accompanying drawings in which:

FIG. 1 shows a side view of an assembled wax cutting tool ready for operation, the wax cutting tool having a cylindrical central rod and a tubular member disposed over the central cylindrical rod, the tubular member being free to rotate and translate between top and bottom subs, the tubular member having a substantially circular cross-section with projections for mounting a wire blade thereto;

FIG. 2 shows various views of the components of the wax cutting tool shown in FIG. 1 (without the wire threaded);

FIG. 3 shows a side view of another assembled wax cutting tool ready for operation, the wax cutting tool having a central cylindrical rod and a tubular member disposed over the central cylindrical rod, the tubular member being free to rotate and translate between top and bottom subs, the tubular member having a substantially square cross-section with a wire blade attached thereto at corners of the square tubular member;

FIG. 4 shows a cross-sectional view of the components of the wax cutting tool shown in FIG. 3 (without the wire blade);

FIG. 5 shows a side view of the components of the wax cutting tool shown in FIG. 3 (without the wire blade);

FIG. 6 shows another cross-sectional view of the components of the wax cutting tool shown in FIG. 3 (without the wire blade); and

FIG. 7 shows more views of the components of the wax cutting tool shown in FIG. 3 in semi-assembled form including the wire blade mounted through corners of the square tubular member as indicated most clearly in the cross-sections.

The following table provides a list of the elements shown in the Figures. Note that the term “sub” is a term of art which means any small component of a downhole assembly.

### DETAILED DESCRIPTION OF THE INVENTION

A wireline wax cutter tool is described herein for detaching a build-up of paraffin wax which has stuck to an oil well tubing internal wall. The wax cutting tool has a top thread A for connecting to a rope socket, wireline tool string, pump rod or any conveyance means for the passage intervention into a well. The tool is made up of a center rod section E, with a top sub C and bottom end cap N. The center rod E has a fixed diameter top to bottom and is parallel along its length. A center sub (tubular member F of cutter body) has a parallel hole from top to bottom which allows movement of the tubular member F of the cutter body about the centerline of the center rod section E. The top thread connector A and bottom end cap N are mechanically attached to the center rod E. Top sub C and bottom end cap N limit the cutter movement up and down and retain the tubular member F of the cutter on the center rod E so that it captured on the center rod E. The tubular member F is free moving up and down the center rod E. The cutter body F includes a tubular section with a number of wings H, J extending outwards increasing its overall diameter. The wings H, J are shaped with a radius of curvature or a beveled angle to stop them becoming hung up on any edges when run in the well. At the out diameter of the wings H, J holes are positioned to retain a wire blade K. The wire blade K is coiled around the cutter body in a helix shape. The outside diameter for the helix is the same and slightly bigger than the well tubing inside diameter. The wire blade helix shape allows compression of the wire blade K and reduces the outside diameter to the same as the tubing wall inside diameter. As the tool is run down within a well the blade K cuts through the wax build up releasing it from the well tubing wall.

The wax cutting tool assembly is adapted to be mounted on any means of well intervention, and is of a type that is self-contained to include a means to provide a jarring action and produce impact, and a low physical cross-sectional area to allow fluid by-pass within a well. The wax cutting action is produced by a low cross-sectional assembly extending to the maximum inside diameter within the well.

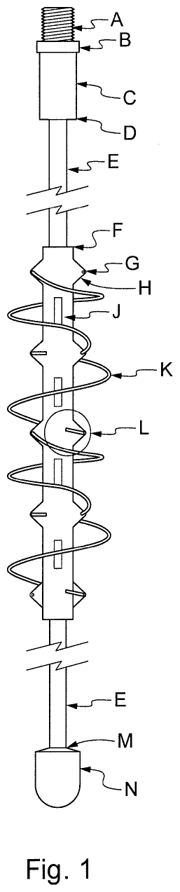

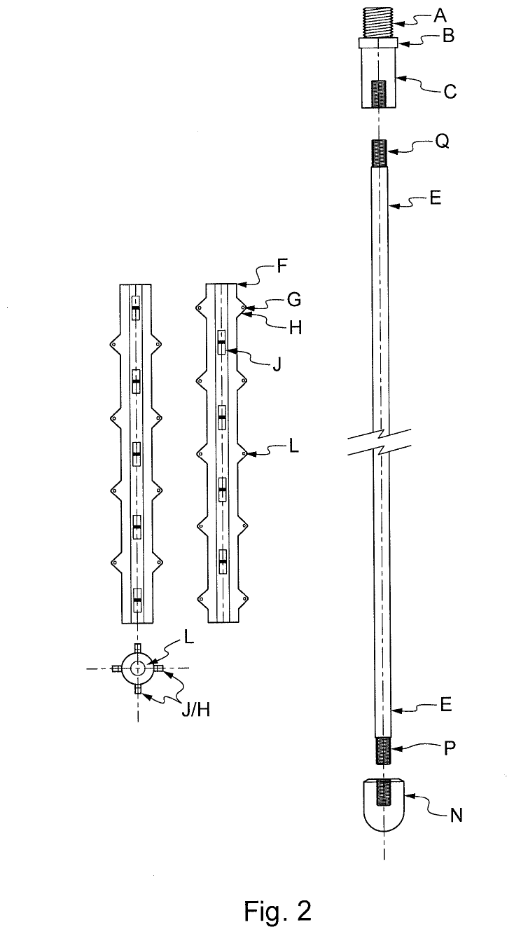

As shown in FIGS. 1 and 2, a wax cutter tool string in made up of the cutter and integral jar device which produces an impact should the cutter body F become held up in operation. The top thread A is used to connect the wax cutting tool to a wireline via a standard rope socket. Should the tool become detached in the well, the fish neck B is used to locate and retrieve the tool as with standard wireline operation. The top cross-over and retainer sub C is used to attach the center rod section E to the wireline rope socket and retain the cutter body F on the center rod section E when in operation. The cutter body F is utilized to detach the wax from the tubing wall by means of a wire, lace, rod, chord, scraper, or blade K. The cutter body F is machined to produce a low cross-sectional area to assist by pass of fluid when within the well bore tubing as this assists the clearing of the wax from the cutting means. The cutting means—wire, lace, rod, or blade K is attached to the cutter body F. The cutting means K is flexible and is sized just larger than the tubing to be wax cut. The cutter means K is shaped in a helical fashion to allow the cutter K to maintain the maximum diameter when in operation. The center body F is free to move up and down the center rod section E and retained by both the top sub C and the bottom sub N. The bottom sub N can be secured to the center rod E by a thread or it can be integral to the center rod E.

A spiral wound wire is used to cut the wax and it has the ability to coil and change diameter and maintain pressure on the inside wall of the tubing being wax cut.

The configuration generations ajar action and jar force from the wax cutting tool moving independently up and down a center element. Should the center wax cutting tool become hung up, the center element is allowed to still move until the wax cutting tool reaches a center element limits (top limit). This will generate the majority of the jar action within the tool when running down within the well bore. It works like a sliding hammer with the center element (rod) still moving when the wax cutting tool becomes held up.

The tool incorporates cutting, sizing, and impact force functionality within the same assemble. Tool length can be reduced by integrating cutting, sizing, and jarring actions. Furthermore, the tool configuration has a low physical cross-sectional area to reduce flow lift from the well and increase fluid bypass in the well.

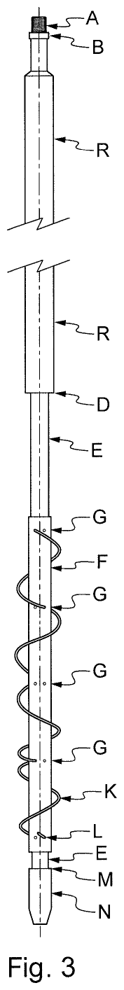

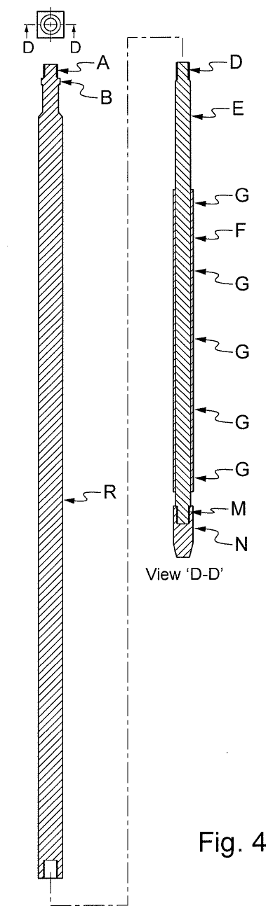

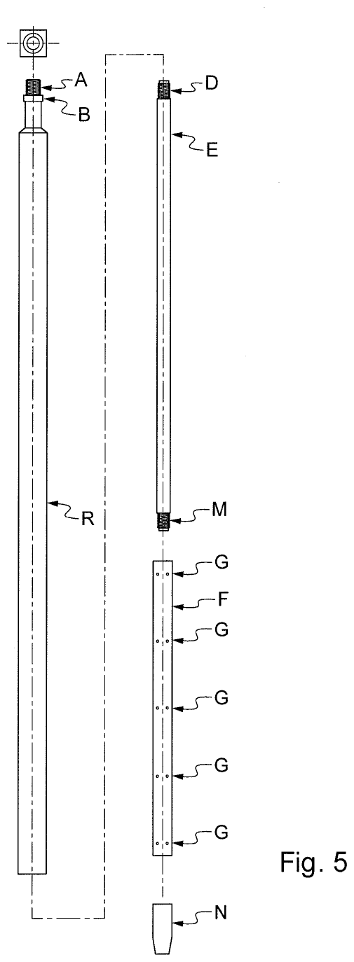

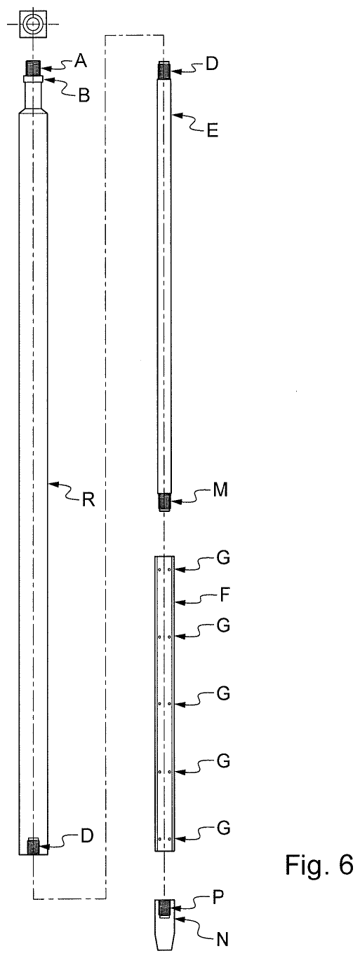

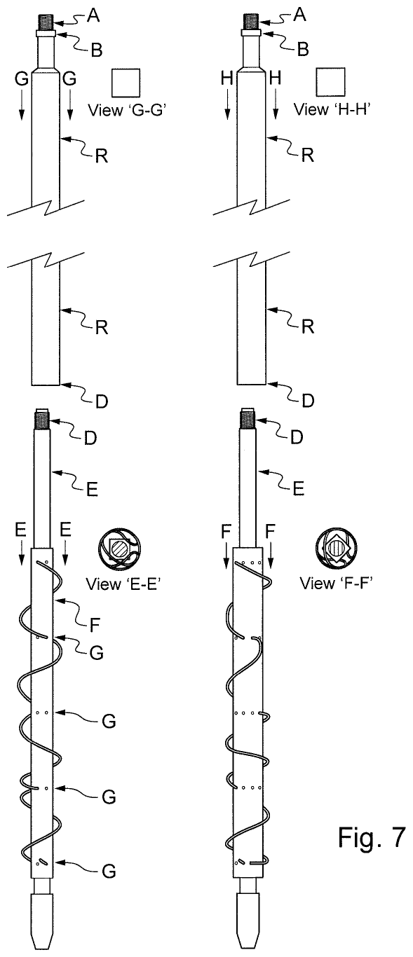

FIGS. 1 and 2 illustrated an embodiment of wax cutter tool in which the tubular member F has a substantially circular cross-section with a number of wings H, J extending outwards increasing its overall diameter. The wings H, J have holes to retain a wire blade K. In contrast, FIGS. 3 to 7 show an alternative embodiment in which the tubular member F has a substantially square cross-section with holes through the corners of the square-shaped tubular member F to retain a wire blade K. Note that like reference numerals have been used for like parts to illustrate the common features of this embodiment with the embodiment illustrated in FIGS. 1 and 2. The exception is reference R which has been used to denote a square stem assembly of the top sub. In this embodiment, the square stem of the top sub has increased weight. The center rod E remains round (circular cross-section) as in the previous embodiment. The round center rod E can be coupled to the square stem of the top sub by connector D which can be, for example, a threaded connector. The square tubular member F fits over the round/cylindrical center rod E and is free to rotate relative to the center rod and to translate up and down the center rod between the top sub and the bottom sub N. The square tubular member F is more simple to manufacture compared with the tubular member with wings of the embodiment illustrated in FIGS. 1 and 2. Furthermore, the square tubular member has a reduced contact area with the cylindrical center rod and thus has reduced friction when compared to a round tubular member. The square tubular member also allows greater bypass of cut wax from the tubing wall and/or fluid flowing through the tubing in which the wax cutting tool is disposed. In yet another alternative (not illustrated) the central rod could be polygonal and the outer tubular member could be round/cylindrical. Alternatively, two (e.g. different) polygonal components could be provided for the central rod and overlying tubular member.

While this invention has been described in relation to embodiments it will be appreciated that various alternative embodiments can be provided. For example, the described embodiments incorporate a combination of: (i) flexible cutting elements enabling the cutting elements to be compressed to change diameter and maintain pressure on the inside wall of the tubing being wax cut; (ii) a low physical cross-sectional area to enable fluid flow and clearing of wax; and (iii) a sliding hammer action to provide a jar force to release the tool if it becomes held up. While it is advantageous to combine these different features, it is also envisaged that they could be provided separately depending on requirements. For example, a wax cutter tool for removing wax from an oil well tubing may be provided, the wax cutter tool comprising: a central wax cutter tool body, the central wax cutter tool body being configured to be attached to an elongate member for deployment down an oil well tubing; and one or more wax cutter elements extending radially outwards from the central wax cutter tool body, the one or more wax cutter elements having an outer working surface for engaging the internal wall of the oil well tubing in use, the outer working surface of the one or more wax cutter elements defining an outer diameter of the wax cutter tool, wherein the wax cutter tool comprises one or more of the following features: the one or more wax cutter elements are flexible thereby allowing compression of the one or more cutter elements in use when deployed within the oil well tubing such that the outer diameter of the outer working surface of the one or more wax cutter elements is reduced to match an inner diameter of the oil well tubing when inserted into the oil well tubing to remove wax from the inner wall of the oil well tubing; one or more fluid paths are provided between the central wax cutter tool body and the outer working surface of the one or more wax cutter elements to enable fluid to flow though the wax cutter tool when it is deployed in an oil well tubing, the wax cutter tool having a total cross-sectional area defined by the outer working surface, and the one or more fluid paths have a cross-sectional area which forms at least 30%, 50%, or 75% of the total cross-sectional area of the wax cutter tool; and the central wax cutter tool body is configured to enable free translational movement of the one or more wax cutter elements between upper and lower stoppers when the wax cutter tool is coupled to an elongate member and deployed down an oil well tubing, the central wax cutter tool body being thus configured to provide a sliding hammer action to generate a jar force to release the wax cutter tool if it becomes held up.

What is claimed is:

1. A wax cutter tool for removing wax from an internal wall of an oil well tubing, the wax cutter tool comprising:
a central wax cutter tool body, the central wax cutter tool body being configured to be attached to an elongate member for deployment down an oil well tubing; and
one or more wax cutter elements extending radially outwards from the central wax cutter tool body, the one or more wax cutter elements having an outer working surface for engaging the internal wall of the oil well tubing in use, the outer working surface of the one or more wax cutter elements defining an outer diameter of the wax cutter tool,
wherein the one or more wax cutter elements are flexible thereby allowing compression of the one or more cutter elements in use when deployed within the oil well tubing such that the outer diameter of the outer working surface of the one or more wax cutter elements is reduced to match an inner diameter of the oil well tubing when inserted into the oil well tubing to remove wax from the internal wall of the oil well tubing.

2. The wax cutter tool of claim 1,
wherein the working surface of the one or more wax cutter elements is flexible to enable compression of the one or more cutter elements in use when deployed within the oil well tubing.

3. The wax cutter tool of claim 1,
wherein the one or more cutter elements includes a wire blade.

4. The wax cutter tool of claim 3,
wherein the one or more cutter elements includes a plurality of wire blades disposed along the central wax cutter tool body.

5. The wax cutter tool of claim 1,
wherein the one or more wax cutter elements form a helical or spiral wire blade.

6. The wax cutter tool of claim 1,
wherein one or more fluid paths are provided between the central wax cutter tool body and the outer working surface of the one or more wax cutter elements to enable fluid to flow though the wax cutter tool when it is deployed in an oil well tubing.

7. The wax cutter tool of claim 6,
wherein the wax cutter tool has a total cross-sectional area defined by the outer working surface, and the one or more fluid paths have a cross-sectional area which forms at least 30%, 50%, or 75% of the total cross-sectional area of the wax cutter tool.

8. The wax cutter tool of claim 1,
wherein the central wax cutter tool body is configured to enable free rotation of the one or more wax cutter elements when the wax cutter tool is coupled to an elongate member and deployed down an oil well tubing.

9. The wax cutter tool according to claim 1,
wherein the central wax cutter tool body is configured to enable free translational movement of the one or more wax cutter elements between upper and lower stoppers when the wax cutter tool is coupled to an elongate member and deployed down an oil well tubing, the central wax cutter tool body being thus configured to provide a sliding hammer action to generate a jar force to release the wax cutter tool if it becomes held up.

10. The wax cutter tool of claim 9,
wherein the central wax cutter body comprises a tubular member and a central rod extending through the tubular member, wherein the tubular member is rotatable around the central rod, and wherein the one or more wax cutter elements are mounted on the tubular member.

11. The wax cutter tool of claim 10,
wherein the tubular member is movable up and down the central rod and the central rod includes an upper stopper and a lower stopper to limit the extent of translational movement of the tubular member.

12. The wax cutter tool of claim 10,
wherein the tubular member has a circular cross-section or a polygonal cross-section.

13. The wax cutter tool of claim 1,
wherein the central wax cutter tool body comprises a plurality of protrusions or corners providing attachment points for the one or more cutter elements.

14. The wax cutter tool of claim 13,
wherein the plurality of protrusions are shaped with a radius of curvature or a bevelled angle.

15. An oil well wax cutter system comprising:
an elongate member for deployment down an oil well tubing; and
a wax cutter tool according to any preceding claim which is configured to be attached to the elongate member for deployment down an oil well tubing.

16. The oil well wax cutter system of claim 15,
wherein the outer diameter of the wax cutter tool is equal to or larger than the inner diameter of the oil well tubing in a fully expanded form and is compressible to be inserted into the oil well tubing.

17. A method of removing wax from an internal wall of an oil well tubing, the method comprising:
connecting the wax cutter tool of claim 1 to an elongate member;
inserting the wax cutter tool into an oil well tubing; and
moving the wax cutter tool along the oil well tubing to remove wax from an inner wall of the oil well tubing.
